(19) United States
(12) Patent Application Publication
(10) Pub. No.: US 2024/0390657 A1
(43) Pub. Date: Nov. 28, 2024

(54) URETERAL STENT AND ITS TESTING DEVICE

(71) Applicant: COPPER MEDICAL TECHNOLOGY CO., LTD., Shenzhen (CN)

(72) Inventors: Yanfa MAO, Shenzhen (CN); Youlu HUA, Shenzhen (CN)

(73) Assignee: COPPER MEDICAL TECHNOLOGY CO., LTD., Shenzhen (CN)

(21) Appl. No.: 18/796,154

(22) Filed: Aug. 6, 2024

Related U.S. Application Data
(63) Continuation of application No. PCT/CN2023/071855, filed on Jan. 12, 2023.

(30) Foreign Application Priority Data
Apr. 13, 2022 (CN) 202210382687.4

(51) Int. Cl. A61M 27/00; A61F 2/04

(52) U.S. Cl. CPC A61M 27/008; A61F 2002/048; A61F 2210/0014; A61M 2205/3334

(57) ABSTRACT

A ureteral stent includes a metal stent and a tubular membrane, the metal stent includes a plurality of unit sections. The application further discloses a testing device, which includes a silicone hose, an outer hard tube, a ureteral stent, a pressure mechanism, a flow mechanism, a flow rate mechanism, a rotation mechanism; the pressure mechanism is connected with the space between the silicone hose and outer hard tube, and the pressure in the pressure mechanism can enable the silicone hose to expand or contract; the flow mechanism can change the speed of the water pump with the frequency converter to adjust the flow rate of liquid in the silicone hose, and the flow rate mechanism can change the shape of the silicone hose to adjust the flow rate of liquid in silicone hose.

### CROSS-REFERENCE TO RELATED APPLICATIONS

This application is a continuation of International Application No. PCT/CN2023/071855, filed on Jan. 12, 2023, which claims priority to Chinese Patent Application No. 202210382687.4, filed on Apr. 13, 2022. All of the aforementioned applications are incorporated herein by reference in their entireties.

### TECHNICAL FIELD

The application relates to the technical field of medical devices, in particular to a ureteral stent and a testing device thereof.

### BACKGROUND

In medical devices, there is a metal stent used to open lumen channels in the body, such as ureter, blood vessel, bile duct, esophagus, etc. The outer layer of the metal stent is generally provided with a plastic film to prevent the metal stent from directly contacting the blood vessel wall. However, there is no special testing device for the process of sticking the metal stent and film together. The film produced by ordinary blow molding is still too thick for medical devices to be used, and the connection process between the film produced by blow molding and the metal stent is very complicated.

If there is something wrong with the human ureter, it will make it difficult for the human body to urinate. Ureteral stent is often implanted to assist urination. Metal ureteral stent is widely used to treat patients with tumor compression or severe ureteral stricture because of its excellent supporting ability and low postoperative stone rate and infection rate. However, the implantation of metal ureteral stent would cause acute injury to the ureteral tissue of patients at early stage, which would promote the deposition of extracellular matrix and form acute stenosis. After the metal ureteral stent is implanted for a period of time, due to the patient's own conditions and the continuous stimulation caused by the metal ureteral stent in the body with the peristalsis of the ureter, it is likely to induce the ureteral tissue to fail to heal, and fibroblasts proliferate excessively to form scar tissue, leading to secondary stenosis. Therefore, there is an urgent need for some tests for ureteral stent to meet the requirements of use.

### SUMMARY

In view of the above problems, the application provides a ureteral stent to solve the problem of ureteral passage support in vivo. The invention also provides a testing device for ureteral stent, which solves the problem of ureteral stent testing.

In order to solve the above technical problems, the technical solution of the application are as follows. A ureteral stent is provided, including a metal stent made of monofilaments and a tubular membrane, the tubular membrane is formed by ultrasonic atomization spraying with film covering solution and then curing; the tubular membrane is arranged outside the metal stent and connected with the metal stent, and the metal stent is in a wave shape with wave crest and wave trough; the metal stent includes a plurality of unit sections, except the first unit section and the last unit section, a tail end of the unit section is connected with a head end of another unit section; or, except the first unit section and the last unit section, a head end of the unit section is connected with a tail end of another unit section; center lines of the metal stents are all on a same cylindrical surface.

An included angle between a plane formed by the head end point of the unit section and a central line of the tubular membrane and a plane formed by the tail end point of the unit section and the central line of the tubular membrane is divided by 360 degrees to obtain a numerical value K1, a distance between the head end point of the unit section and the tail end point of the unit section in a central line direction of the tubular membrane is divided by a distance between the highest point of the wave crest and the lowest point of the wave trough in the central line direction of the tubular membrane to obtain a numerical value; K1=n*K2, so that for the plurality of unit sections, wave crest corresponds to wave crest, wave trough corresponds to wave trough, and n is a natural integer between 0 and 10.

As a preferred solution of the application, the metal stent is made of metallic titanium or nickel-titanium alloy.

As a preferred solution of the application, the metal stent is made of round nickel-titanium wire.

As a preferred solution of the application, the tubular membrane is a polycarbonate polyurethane film.

A testing device of ureteral stent is provided, including a silicone hose for simulating ureter, an outer hard tube for simulating ureter expansion and contraction, an ureteral stent, a pressure mechanism for expanding and contracting the silicone hose, a flow mechanism for controlling liquid flow in the silicone hose, a flow rate mechanism for controlling liquid flow rate in the silicone hose, a rotation mechanism for simulating human body state; wherein the pressure mechanism is connected with a space between the silicone hose and the outer hard tube, and a pressure in the pressure mechanism is able to expand or contract the silicone hose; the flow mechanism is able to change a rotating speed of a water pump using a frequency converter to change a flow rate of liquid in the silicone hose, and the flow rate mechanism is able to change a shape of the silicone hose to change the flow rate of liquid in the silicone hose; the ureteral stent is placed in the silicone hose, the outer hard tube is placed outside the silicone hose, the water pump is connected with the silicone hose, the flow rate mechanism is arranged outside the silicone hose; and the silicone hose, the outer hard tube and the ureteral stent are arranged on the rotation mechanism.

Preferably, the rotation mechanism includes a first rotation motor, a second rotation motor, a lifting ring, a first rotation ring, a second rotation ring, the lifting ring is installed on an equipment bracket, the first rotation motor is installed on the lifting ring, and the first rotation ring is installed on the first rotation motor; the second rotation motor is installed on the first rotation ring, the second rotation ring is installed on the second rotation motor, and the second rotation ring is provided with a plurality of test units.

Preferably, the testing unit includes a silicone hose, an outer hard tube, a ureteral stent, a first outer-tube sealing part, and a second outer-tube sealing part; the first outer-tube sealing part is provided with a hose hole for the silicone hose to pass through and an air pressure hole for external pressure mechanism, the second outer-tube sealing part is provided with a hose hole for the silicone hose to pass through; the silicone hose passes through the hose hole of the first outer-tube sealing part and is hermetically connected with the silicone hose and the first outer-tube sealing part; the silicone hose passes through the hose hole of the second outer-tube sealing part and is hermetically connected with the silicone hose and the second outer-tube sealing part; the air pressure hole is connected with the pressure mechanism; the first outer-tube sealing part and the second outer-tube sealing part are respectively arranged at two ends of the outer hard tube; the first outer-tube sealing part and the second outer-tube sealing part are hermetically connected with the outer hard tube, and a closed space is formed among the first outer-tube sealing part, the second outer-tube sealing part, the silicone hose and the outer hard tube.

Preferably, the pressure mechanism includes a cylinder, a pressure motor; a cavity of the cylinder is connected with the closed space formed by the first outer-tube sealing part, the second outer-tube sealing part, the silicone hose and the outer hard tube; a piston of the cylinder is connected with the pressure motor, the pressure motor drives the piston of the cylinder to slide in the cavity of the cylinder to allow the pressure mechanism to be transferred to the closed space formed by the first outer-tube sealing part, the second outer-tube sealing part, the silicone hose and the outer hard tube, so as to enable the silicone hose to contract or expand.

Preferably, the flow mechanism includes a frequency converter and a water pump, and the frequency converter is able to change a rotation speed of the water pump to adjust a flow rate of the water pump.

Preferably, the flow rate mechanism includes a flow rate motor, a bottom plate, a clamp plate and a guide groove, and the bottom plate is fixedly connected with the guide groove; the clamp plate is slidable in the guide groove, and the flow rate motor drives the clamp plate to slide in the guide groove relative to the bottom plate, so as to change a cross-sectional area of the silicone hose to adjust the flow rate.

The above technical solutions have the following advantages. The polycarbonate polyurethane film produced by spraying is much thinner than that produced by blow molding, and the thickness of the film produced by spraying in this application may be between 0.025 and 0.05 mm, which solves the problem that small-diameter film tubes cannot be produced by blow molding and the problem of connection between the tubular membrane and metal stent.

In this application, the silicone hose, outer hard tube, pressure mechanism, flow mechanism, flow rate mechanism and rotation mechanism are adopted to simulate the use environment of the ureteral stent for testing, thus ensuring the product quality of the ureteral stent.

### BRIEF DESCRIPTION OF THE DRAWINGS

In order to more clearly explain the embodiments of the present application or the technical solution in the prior art, the drawings mentioned in the embodiments will be briefly introduced below. Apparently, the drawings described below illustrate only some embodiments of the present application. For those of ordinary skill in this field, other drawings may be obtained according to these drawings without any creative effort.

FIG. 1 is a perspective view of a ureteral stent.

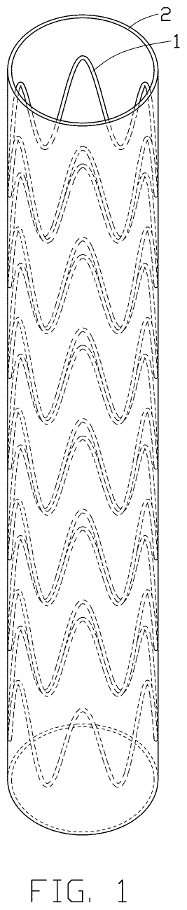

FIG. 2 is a perspective view of a metal stent.

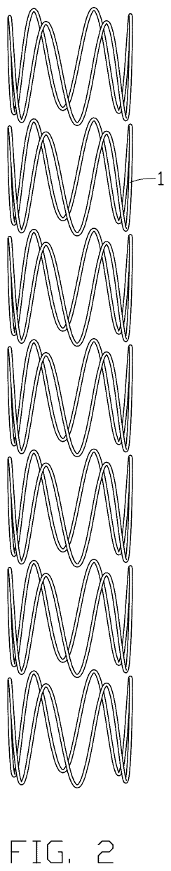

FIG. 3 is a perspective view of the present application.

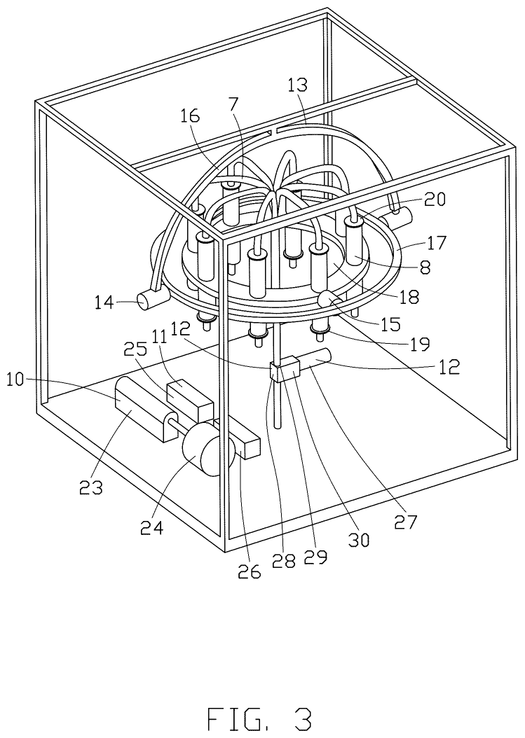

FIG. 4 is another perspective view of the application.

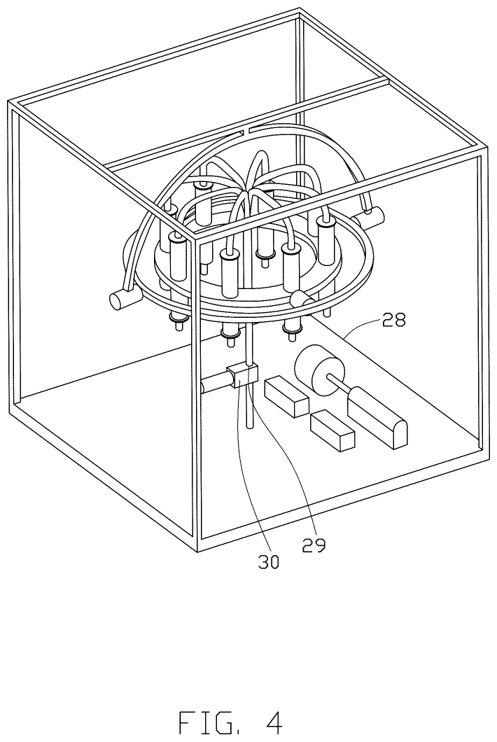

FIG. 5 is a perspective view of a test unit.

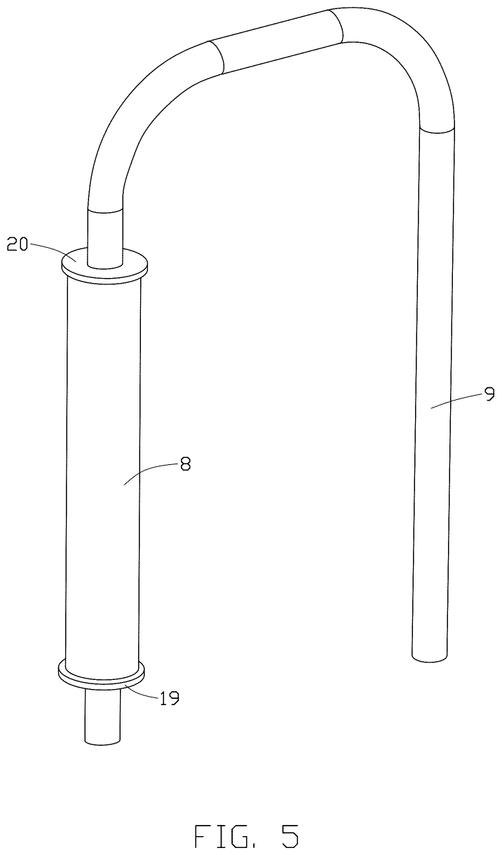

FIG. 6 is an exploded view of a test unit.

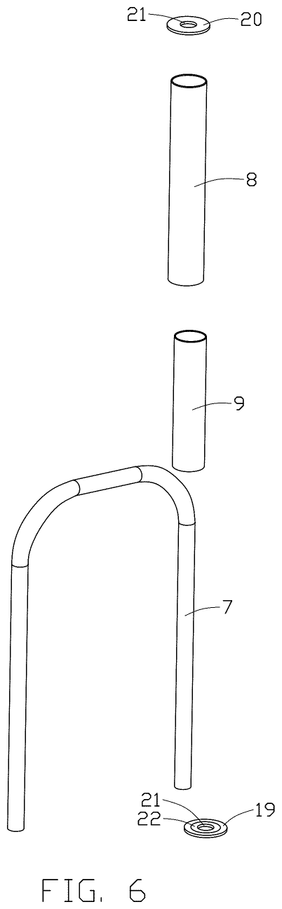

FIG. 7 is a schematic diagram of a unit section.

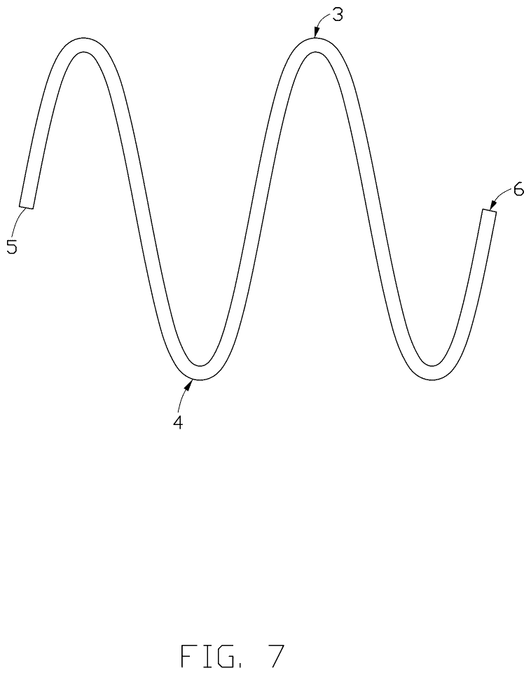

Reference signs in the figures are as below.
1—Metal stent; 2—Tubular membrane; 3—Wave crest; 4—Wave trough; 5—Head end; 6—Tail end; 7—Silicone hose; 8—Outer hard tube; 9—Ureteral stent; 10—Pressure mechanism; 11—Flow mechanism; 12—Flow rate mechanism; 13—Rotation mechanism; 14—First rotation motor; 15—Second rotation motor; 16—Lifting ring; 17—First rotation ring; 18—Second rotation ring; 19—First outer-Tube sealing part; 20—Second outer-Tube sealing part; 21—Hose hole; 22—Air pressure hole; 23—Cylinder; 24—Pressure motor; 25—Water pump; 26—Frequency converter; 27—Flow rate motor; 28—Bottom plate; 29—Clamp plate; 30—Guide groove.

### DETAILED DESCRIPTION OF THE EMBODIMENTS

In order to further illustrate the embodiments, the application provides the attached drawings, which are a part of the disclosure of the application, and are mainly used to illustrate the embodiments and explain the working principle of the embodiments with the relevant descriptions in the specification. With reference to these contents, the ordinary person in the field would be able to understand other possible embodiments and the advantages of the application. Components in the drawings are not drawn to scale, and one reference sign generally indicate one kind of component.

The specific embodiments of the present application will be further described with reference to the attached drawings. It should be noted here that the description of these embodiments is used to help understand the application, but it does not constitute a limitation of the application. In addition, the technical features involved in embodiments of the application described below can be combined with each other as long as they do not conflict with each other.

Referring to the attached drawings, a ureteral stent 9 is provided, including a metal stent 1 made of monofilaments and a tubular membrane 2, the tubular membrane 2 is formed by ultrasonic atomization spraying with film covering solution and then curing; the tubular membrane 2 is arranged outside the metal stent 1 and connected with the metal stent 1, and the metal stent 1 is in a wave shape with wave crest 3 and wave trough 4; the metal stent 1 includes a plurality of unit sections, except the first unit section and the last unit section, a tail end 6 of the unit section is connected with a head end 5 of another unit section; or, except the first unit section and the last unit section, a head end 5 of the unit section is connected with a tail end 6 of another unit section; center lines of the metal stents 1 are all on a same cylindrical surface.

An included angle between a plane formed by the point of head end 5 of the unit section and a central line of the tubular membrane 2 and a plane formed by the point of tail end 6 of the unit section and the central line of the tubular membrane 2 is divided by 360 degrees to obtain a numerical value K1, a distance between the point of head end 5 of the unit section and the point of tail end 6 of the unit section in a central line direction of the tubular membrane 2 is divided by a distance between the highest point of the wave crest 3 and the lowest point of the wave trough 4 in the central line direction of the tubular membrane to obtain a numerical value; K1=n*K2, so that for the plurality of unit sections, wave crest 3 corresponds to wave crest 3, wave trough 4 corresponds to wave trough 4, and n is a natural integer between 0 and 10. The metal stent 1 made of monofilaments refers to a metal stent 1 made of a metal wire.

Preferably, the metal stent 1 is made of metallic titanium or nickel-titanium alloy.

Preferably, the metal stent 1 is made of round nickel-titanium wire.

Preferably, the tubular membrane 2 is a polycarbonate polyurethane film.

A testing device of ureteral stent is provided, including a silicone hose 7 for simulating ureter, an outer hard tube 8 for simulating ureter expansion and contraction, an ureteral stent 9, a pressure mechanism 10 for expanding and contracting the silicone hose 7, a flow mechanism 11 for controlling liquid flow in the silicone hose 7, a flow rate mechanism 12 for controlling liquid flow rate in the silicone hose 7, a rotation mechanism 13 for simulating human body state; wherein the pressure mechanism 10 is connected with a space between the silicone hose 7 and the outer hard tube 8, and a pressure in the pressure mechanism 10 is able to expand or contract the silicone hose 7; the flow mechanism 11 is able to change a rotating speed of a water pump using a frequency converter to change a flow rate of liquid in the silicone hose 7, and the flow rate mechanism 12 is able to change a shape of the silicone hose 7 to change the flow rate of liquid in the silicone hose 7; the ureteral stent 9 is placed in the silicone hose 7, the outer hard tube 8 is placed outside the silicone hose 7, the water pump is connected with the silicone hose 7, the flow rate mechanism 12 is arranged outside the silicone hose 7; and the silicone hose 7, the outer hard tube 8 and the ureteral stent 9 are arranged on the rotation mechanism 13.

Preferably, the rotation mechanism 13 includes a first rotation motor 14, a second rotation motor 15, a lifting ring 16, a first rotation ring 17, a second rotation ring 18, the lifting ring 16 is installed on an equipment bracket, the first rotation motor 14 is installed on the lifting ring 16, and the first rotation ring 17 is installed on the first rotation motor 14; the second rotation motor 15 is installed on the first rotation ring 17, the second rotation ring 18 is installed on the second rotation motor 15, and the second rotation ring 18 is provided with a plurality of test units.

Preferably, the testing unit includes a silicone hose 7, an outer hard tube 8, a ureteral stent 9, a first outer-tube sealing part 19, and a second outer-tube sealing part 20; the first outer-tube sealing part 19 is provided with a hose hole 21 for the silicone hose 7 to pass through and an air pressure hole 22 for external pressure mechanism, the second outer-tube sealing part 20 is provided with a hose hole for the silicone hose 7 to pass through; the silicone hose 7 passes through the hose hole 21 of the first outer-tube sealing part 19 and is hermetically connected with the silicone hose 7 and the first outer-tube sealing part 19; the silicone hose 7 passes through the hose hole 21 of the second outer-tube sealing part 20 and is hermetically connected with the silicone hose 7 and the second outer-tube sealing part 20; the air pressure hole 22 is connected with the pressure mechanism; the first outer-tube sealing part 19 and the second outer-tube sealing part 20 are respectively arranged at two ends of the outer hard tube 8; the first outer-tube sealing part 19 and the second outer-tube sealing part 20 are hermetically connected with the outer hard tube 8, and a closed space is formed among the first outer-tube sealing part 19, the second outer-tube sealing part 20, the silicone hose 7 and the outer hard tube 8.

“The flow rate mechanism 12 can change the shape of silicone hose 7 to adjust the flow rate of liquid in silicone hose 7”. Because the cross-sectional area of silicone hose 7 is the largest when it is circular, and the flow rate is the smallest at this state. When the silicone hose 7 is squished, the distance between the two squished sides is proportional to the cross-sectional area of the silicone hose 7. If the distance between the inner walls of silicone hose 7 is zero when the silicone hose 7 is squished, then silicone hose 7 can be closed. If the distance between the inner walls of silicone hose 7 is small when the silicone hose 7 is squished, the flow rate is large. If the distance between the inner walls of silicone hose 7 is large when the silicone hose 7 is squished, the flow rate is small.

Preferably, the pressure mechanism 10 includes a cylinder 23, a pressure motor 24; a cavity of the cylinder 23 is connected with the closed space formed by the first outer-tube sealing part 19, the second outer-tube sealing part 20, the silicone hose 7 and the outer hard tube 8; a piston of the cylinder 23 is connected with the pressure motor 24, the pressure motor 24 drives the piston of the cylinder 23 to slide in the cavity of the cylinder 23 to allow the pressure mechanism 10 to be transferred to the closed space formed by the first outer-tube sealing part 19, the second outer-tube sealing part 20, the silicone hose 7 and the outer hard tube 8, so as to enable the silicone hose 7 to contract or expand.

Preferably, the flow mechanism 11 includes a frequency converter 26 and a water pump 25, and the frequency converter 26 is able to change a rotation speed of the water pump 25 to adjust a flow rate of the water pump 25.

Preferably, the flow rate mechanism 12 includes a flow rate motor 27, a bottom plate 28, a clamp plate 29 and a guide groove 30, and the bottom plate 28 is fixedly connected with the guide groove 30; the clamp plate 29 is slidable in the guide groove 30, and the flow rate motor 27 drives the clamp plate 29 to slide in the guide groove 30 relative to the bottom plate, so as to change a cross-sectional area of the silicone hose 7 to adjust the flow rate.

The polycarbonate polyurethane film produced by spraying is much thinner than that produced by blow molding, and the thickness of the film produced by spraying in this application may be between 0.025 and 0.05 mm, which solves the problem that small-diameter film tubes cannot be produced by blow molding and the problem of connection between the tubular membrane 2 and metal stent 1.

The tubular membrane 2 on the metal stent 1 has a diameter of less than 10 mm; blow molding is unable to produce a membrane with such a tiny diameter or the necessary thickness. It can be created, nevertheless, by spraying, and the thickness created by spraying might range from 0.025 to 0.05 mm.

In this application, the silicone hose 7, outer hard tube 8, pressure mechanism 10, flow mechanism 11, flow rate mechanism 12, rotation mechanism 13 are provided to simulate the use environment of the ureteral stent 9 for testing, significantly enhancing and guaranteeing the product quality of ureteral stent. The present application has the following advantages.

Firstly, using rotation mechanism 13 to simulate the state of human body, such as lying on one side and lying on one's back.

Secondly, flow mechanism 11 and flow rate mechanism 12 are used to simulate the state that ureteral stent 9 faces when the human body urinates.

Thirdly, pressure mechanism 10 is used to simulate the pressure state faced by ureteral stent 9 when the human body urinates.

Fourthly, using silicone hose 7 to simulate ureter to test the cooperation between ureteral stent 9 and ureter.

Fifthly, the film is produced by spraying, which solves the problem that the film tube with a diameter of 10 mm cannot be produced by blow molding.

Sixthly, the tubular membrane 2 is created by spraying, eliminating the need to link it to the metal stent 1. This allows for a direct connection between the tubular membrane 2 and the stent 1.

Lastly, metal stent 1 is made of monofilaments. Compared to multiple metal wires, monofilaments are easier to produce and offer better size control for the metal stent 1.

The embodiments of the present application have been described in detail with the attached drawings, but the application is not limited to the described embodiments. For those skilled in the art, many changes, modifications, substitutions and variations may be made to these embodiments without departing from the principle and spirit of this application, and still fall within the protection scope of this application.

What is claimed is:

1. A ureteral stent, comprising a metal stent made of monofilaments and a tubular membrane, wherein
the tubular membrane is formed by ultrasonic atomization spraying with film covering solution and then curing; the tubular membrane is arranged outside the metal stent and connected with the metal stent, and the metal stent is in a wave shape with wave crest and wave trough; the metal stent comprises a plurality of unit sections, except the first unit section and the last unit section, a tail end of the unit section is connected with a head end of another unit section; or, except the first unit section and the last unit section, a head end of the unit section is connected with a tail end of another unit section; center lines of the metal stents are all on a same cylindrical surface; and
an included angle between a plane formed by the head end point of the unit section and a central line of the tubular membrane and a plane formed by the tail end point of the unit section and the central line of the tubular membrane is divided by 360 degrees to obtain a numerical value K1, a distance between the head end point of the unit section and the tail end point of the unit section in a central line direction of the tubular membrane is divided by a distance between the highest point of the wave crest and the lowest point of the wave trough in the central line direction of the tubular membrane to obtain a numerical value; K1=n*K2, so that for the plurality of unit sections, wave crest corresponds to wave crest, wave trough corresponds to wave trough, and n is a natural integer between 0 and 10.

2. The ureteral stent of claim 1, wherein the metal stent is made of metallic titanium or nickel-titanium alloy.

3. The ureteral stent of claim 1, wherein the metal stent is made of round nickel-titanium wire.

4. The ureteral stent of claim 1, wherein the tubular membrane is a polycarbonate polyurethane film.

5. A testing device of ureteral stent, comprising a silicone hose for simulating ureter, an outer hard tube for simulating ureter expansion and contraction, an ureteral stent, a pressure mechanism for expanding and contracting the silicone hose, a flow mechanism for controlling liquid flow in the silicone hose, a flow rate mechanism for controlling liquid flow rate in the silicone hose, a rotation mechanism for simulating human body state;
wherein the pressure mechanism is connected with a space between the silicone hose and the outer hard tube, and a pressure in the pressure mechanism is able to expand or contract the silicone hose; the flow mechanism is able to change a rotating speed of a water pump using a frequency converter to change a flow rate of liquid in the silicone hose, and the flow rate mechanism is able to change a shape of the silicone hose to change the flow rate of liquid in the silicone hose; the ureteral stent is placed in the silicone hose, the outer hard tube is placed outside the silicone hose, the water pump is connected with the silicone hose, the flow rate mechanism is arranged outside the silicone hose, and the silicone hose; the outer hard tube and the ureteral stent are arranged on the rotation mechanism.

6. The testing device of claim 5, wherein the rotation mechanism comprises a first rotation motor, a second rotation motor, a lifting ring, a first rotation ring, a second rotation ring, the lifting ring is installed on an equipment bracket, the first rotation motor is installed on the lifting ring, and the first rotation ring is installed on the first rotation motor; the second rotation motor is installed on the first rotation ring, the second rotation ring is installed on the second rotation motor, and the second rotation ring is provided with a plurality of test units.

7. The testing device of claim 6, wherein the testing unit comprises a silicone hose, an outer hard tube, a ureteral stent, a first outer-tube sealing part, and a second outer-tube sealing part; the first outer-tube sealing part is provided with a hose hole for the silicone hose to pass through and an air pressure hole for external pressure mechanism, the second outer-tube sealing part is provided with a hose hole for the silicone hose to pass through; the silicone hose passes through the hose hole of the first outer-tube sealing part and is hermetically connected with the silicone hose and the first outer-tube sealing part; the silicone hose passes through the hose hole of the second outer-tube sealing part and is hermetically connected with the silicone hose and the second outer-tube sealing part; the air pressure hole is connected with the pressure mechanism; the first outer-tube sealing part and the second outer-tube sealing part are respectively arranged at two ends of the outer hard tube; the first outer-tube sealing part and the second outer-tube sealing part are hermetically connected with the outer hard tube, and a closed space is formed among the first outer-tube sealing part, the second outer-tube sealing part, the silicone hose and the outer hard tube.

8. The testing device of claim 5, wherein the pressure mechanism comprises a cylinder, a pressure motor; a cavity of the cylinder is connected with the closed space formed by the first outer-tube sealing part, the second outer-tube sealing part, the silicone hose and the outer hard tube; a piston of the cylinder is connected with the pressure motor, the pressure motor drives the piston of the cylinder to slide in the cavity of the cylinder to allow the pressure mechanism to be transferred to the closed space formed by the first outer-tube sealing part, the second outer-tube sealing part, the silicone hose and the outer hard tube, so as to enable the silicone hose to contract or expand.

9. The testing device of claim 5, wherein the flow mechanism comprises a frequency converter and a water pump, and the frequency converter is able to change a rotation speed of the water pump to adjust a flow rate of the water pump.

10. The testing device of claim 5, wherein the flow rate mechanism comprises a flow rate motor, a bottom plate, a clamp plate and a guide groove, and the bottom plate is fixedly connected with the guide groove; the clamp plate is slidable in the guide groove, and the flow rate motor drives the clamp plate to slide in the guide groove relative to the bottom plate, so as to change a cross-sectional area of the silicone hose to adjust the flow rate.
